(19) United States
(12) Patent Application Publication
(10) Pub. No.: US 2023/0190126 A1
(43) Pub. Date: Jun. 22, 2023

(54) SYSTEM AND METHOD FOR DETERMINING A PHYSIOLOGICAL PARAMETER ASSOCIATED WITH AN ANATOMICAL CAVITY

(71) Applicant: THE ROYAL INSTITUTION FOR THE ADCANCEMENT OF LEARNING/McGILL UNIVERSITY, Montreal (CA)

(72) Inventors: Mark DRISCOLL, Montreal (CA); Natasha JACOBSON, Montreal (CA)

(21) Appl. No.: 17/926,834

(22) PCT Filed: May 21, 2021
(86) PCT No.: PCT/CA2021/050696

§ 371 Date: Nov. 21, 2022

Related U.S. Application Data
(60) Provisional application No. 63/028,241, filed on May 21, 2020.

(51) Int. Cl. A61B 5/03; A61B 5/00

(52) U.S. Cl. CPC A61B 5/03; A61B 5/0053

(57) ABSTRACT

A method for determining a anatomical cavity pressure comprising; obtaining a pressure value comprising a pressure applied to skin of the anatomical cavity by a vessel, the vessel having a first open end and a second end, the first end arranged to contact the skin to encase a portion of the skin, and the vessel being arranged such that a pressure can be applied through the vessel to the encased portion of the skin; obtaining a skin displacement value associated with the obtained pressure value, the skin displacement value comprising a distance of the encased portion of the skin from a baseline while the pressure is being applied; determining, by the processor, a pressure of the anatomical cavity using the obtained pressure value and the obtained skin displacement value, and based on a static force balance of the vessel and the encased portion of the skin while the pressure is being applied.

### FIELD OF THE DISCLOSURE

The present disclosure relates to a system and a method for determining a physiological parameter associated with an anatomical cavity, such as but not limited to pressure within the anatomical cavity.

### BACKGROUND OF THE DISCLOSURE

Anatomical cavities of humans and other animals include the abdominopelvic cavity (“abdomen”), the thoracic cavity, the cranial cavity, the vertebral cavity, the pericardial cavity, the pleural cavity, muscular cavities and the mediastinum.

In the case of the abdomen, physiological parameters within the abdomen can provide an indication of various abdominal conditions potentially related to organs within the cavity as well as the abdominal muscles. Two such physiological parameters associated with abdominal conditions are intraabdominal pressure (IAP) and abdominal compliance (Cab). Cab, clinically, is the measure of ease of abdominal expansion.

Currently, IAP can only be measured by a direct pressure reading using microtransducers embedded under the abdominal wall to measure intra-peritoneal pressure (IPP), which is synonymous with IAP. A known volume of fluid (e.g. air, saline) is injected into a closed abdominal space (e.g. bladder, stomach, rectum, uterus, or central venous system). The resulting pressure is then measured (via transducer, manometer, or strain gauge) and related to IAP for relevant diagnostics.

However, this an invasive method and carries risk of infection for the patient, as well as discomfort and pain. Furthermore, readings from the microtransducers are position dependent and therefore unreliable. This direct form of IAP measurement is also sensitive to procedural discrepancies such as diaphragm position, patient position, the amount of saline injected, or time before pressure reading.

Therefore, there is a need for systems and methods for determining physiological parameters associated with anatomical cavities which overcome or reduce at least some of the above-described problems.

### SUMMARY OF THE DISCLOSURE

It is an object of the present disclosure to ameliorate at least some of the inconveniences present in the prior art.

Inventors of the present technology have noted certain disadvantages of prior art systems for determining physiological parameters associated with an anatomical cavity. As noted above, the most prevalent technique for determining pressure within the abdomen, is an invasive technique with its associated dangers to the patient.

Inventors have noted that other measurement techniques, which are less invasive, have been proposed but suffer from a wide range of disadvantages including reliability, cost, portability, and sensitivity.

For example:
An ultrasound guided tonometry (UGT) technique has low resolution readings in the form of normal, high and very high.
A skin indentation technique which correlates AWT with IAP can only provide discontinuous readings relating to superficial tissue layers.
A bioimpedance technique correlates the impedance of the abdominal wall with IAP but has low sensitivity.
A microwave reflection technique correlates a reflection coefficient between an antenna and the abdominal wall with IAP, but has a limited pressure range.

According to certain aspects and embodiments of the present technology, a system and method is provided which can determine physiological parameters associated with an anatomical cavity. The system and method is non-invasive. According to certain embodiments, the determination of the parameters is continuous and can therefore be used for monitoring a patient. The system is not prohibitively expensive or complex, and is easy to use. Pressure determination over a broad and physiologically relevant range can be obtained. In certain embodiments, the anatomical cavity is an abdomen of a patient and the pathological parameter is the pressure inside the anatomical cavity. In other embodiments, methods and systems of the present technology may be applied to any other anatomical cavity such as the thoracic cavity, the cranial cavity, the vertebral cavity, the pericardial cavity, the pleural cavity, muscular cavities and the mediastinum.

From one aspect, there is provided a method for determining a pressure within an anatomical cavity of a patient, the method being executed by a processor, the method comprising: obtaining, by the processor, a pressure value, the pressure value comprising a pressure applied to skin associated with the anatomical cavity by a vessel, the vessel having a first end and a second end, the first end being open and arranged to contact the skin to encase a portion of the skin, and the vessel being arranged such that a pressure can be applied through the vessel to the encased portion of the skin; obtaining, by the processor, a skin displacement value associated with the obtained pressure value, the skin displacement value comprising a distance of the encased portion of the skin from a baseline while the pressure is being applied; determining, by the processor, a pressure of the anatomical cavity using the obtained pressure value and the obtained skin displacement value, and based on a static force balance of the vessel and the encased portion of the skin while the pressure is being applied.

In certain embodiments, the static force balance of the vessel and the encased portion of the skin while the pressure is being applied is based on a model of a thick-walled cylinder.

In certain embodiments, the model for a supine anatomical cavity is defined as:

$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )}$$

where Pᵢₙ is the internal vessel pressure, Pₐₚₚ is the applied pressure, r₁ and r₂ are the inner and outer curve radii, respectively, a is the vessel radius, w is the skin displacement value, and t is tissue thickness.

The model is defined, for body positions other than supine, as:

$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )} + ρ g h$$

where ρ is the density of the fluid, g is the force of gravity, and h is the height of the centroid of the anatomical cavity being tested.

In certain embodiments, the local elasticity in a wall of the anatomical cavity is determined using:

$$E = \frac{α ( ζ , ν ) 3 ϕ ( η ) ( {P}_{a t m} - {P}_{a p p} ) a}{2 π w}$$

where E is Young's Modulus, a(ζ,v) is a coefficient dependant on the ratio of tissue thickness to vessel inner radius (ζ=t/r₁) and Poisson's ratio (v), ϕ(η) is a geometric coefficient, and Pₐₜₘ is atmospheric pressure.

In certain other embodiments, the method comprises determining elasticity from a measure of bioimpedance of the skin.

In certain embodiments, obtaining the pressure value comprises the processor obtaining data from a pressure sensor measuring pressure within the vessel and communicatively connected to the processor.

In certain embodiments, obtaining the skin displacement value comprises the processor obtaining data from a distance sensor measuring the displacement of the encased portion of the skin in the vessel and communicatively connected to the processor.

In certain embodiments, the determining the pressure of the anatomical cavity is based on the applied pressure to the encased portion of the skin being between −300 mmHg and 300 mmHg, or between −6 psi and 6 psi.

In certain embodiments, the method further comprises causing, by the processor, a pressure unit connected to the vessel to apply the pressure to the encased portion of the skin through the vessel.

In certain embodiments, the applied pressure is between about −300 mmHg and about 300 mmHg, or between about −6 psi and about 6 psi.

In certain embodiments, the method further comprises causing, by the processor, the vessel to contact the skin before the pressure is applied.

In certain embodiments, the anatomical cavity is an abdomen of a patient.

In certain embodiments, the applied pressure is a negative pressure applied through an opening at the second end of the vessel, and the skin displacement comprises a distance of the encased portion of the skin from the baseline towards the second end of the vessel.

In certain embodiments, the applied pressure is a positive pressure.

From another aspect, there is provided a system for determining a pressure within an anatomical cavity of a patient, the system comprising a processor arranged to execute a method, the method comprising: obtaining, by the processor, a pressure value, the pressure value comprising a pressure applied to an encased portion of skin of the patient associated with the anatomical cavity through a vessel, the vessel having a first end and a second end, the first end being open and arranged to contact the skin to encase a portion of the skin, wherein the vessel is arranged such that a pressure can be applied through the vessel to the encased portion of the skin; obtaining, by the processor, a skin displacement value associated with the obtained pressure value, the skin displacement value comprising a distance of the encased portion of the skin from a baseline while the pressure is being applied; determining, by the processor, a pressure of the anatomical cavity using the obtained pressure value and the obtained skin displacement value, and based on a static force balance of the vessel and the encased portion of the skin while the pressure is being applied.

In certain embodiments, the system further comprises the vessel.

In certain embodiments, the system further comprises a pressure unit fluidly connectable to the vessel for applying the pressure to the encased portion of the skin.

In certain embodiments, the pressure unit comprises a pump.

In certain embodiments, the pressure is a negative pressure and the pressure unit is fluidly connected to the second end of the vessel through which fluid can be drawn out of the vessel to apply the negative pressure to the encased portion of the skin.

In certain embodiments, the pressure unit is arranged to apply a pressure to the encased portion of the skin of between about −300 mmHg and about 300 mmHg, or between about −6 psi and about 6 psi.

In certain embodiments, the vessel has an internal diameter at the first end of about 5-10 cm. In certain embodiments, the internal diameter is more than about 1 cm, more than about 2 cm, more than about 3 cm, between about 1 cm and about 30 cm, between about 2 cm and about 30 cm, or between about 3 cm and about 30 cm.

In certain embodiments, the vessel has an internal diameter at the first end which can be modulated by a shutter-like mechanism.

In certain embodiments, the vessel has a length which can be modulated by a telescoping-like mechanism.

In certain embodiments, the vessel and the pressure unit are encased in an outer casing, the outer casing having a cap portion for closing a second end of the outer casing.

In certain embodiments, the system further comprises a panel attached to the outer casing for user interaction.

In certain embodiments, a length of the vessel is at least equal to, or more than, an internal radius of the vessel.

In certain embodiments, the system further comprises a pressure sensor for measuring the applied pressure in the vessel, the pressure sensor communicatively connected to the processor.

In certain embodiments, the system further comprises a distance sensor for measuring the displacement of the skin while the pressure is being applied, the distance sensor being communicatively connected to the processor.

In certain embodiments, the static force balance of the vessel and the skin while the pressure is being applied is based on a model of a thick-walled cylinder.

In certain embodiments, the model, for an anatomical cavity in a supine position, is defined as:

$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )}$$

where Pᵢₙ is the internal vessel pressure, Pₐₚₚ is the applied pressure, r₁ and r₂ are the inner and outer curve radii, respectively, a is the vessel radius, w is the skin displacement value, and t is tissue thickness.

The model at body positions other than supine introduces a fluid pressure factor, such that it is defined as:

$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )} + ρ g h$$

where ρ is the density of the fluid, g is the force of gravity, and h is the height of the centroid of the anatomical cavity being tested.

In the certain embodiments, a local elasticity in a wall of the anatomical cavity is determined using:

$$E = \frac{α ( ζ , ν ) 3 ϕ ( η ) ( {P}_{atm} - {P}_{a p p} ) a}{2 π w}$$

where E is Young's Modulus, a(ζ,v) is a coefficient dependant on the ratio of tissue thickness to vessel inner radius (ζ=t/r₁) and Poisson's ratio (v), ϕ(η) is a geometric coefficient, and Pₐₜₘ is atmospheric pressure.

In certain other embodiments, the method comprises determining elasticity from a measure of bioimpedance of the skin.

In certain embodiments, the applied pressure is between about −300 mmHg and about 300 mmHg, or between about −6 psi and about 6 psi.

In certain embodiments, the anatomical cavity is an abdomen of a patient.

In certain embodiments, the applied pressure is a negative pressure applied through an opening at the second end of the vessel.

In certain embodiments, the applied pressure is a positive pressure.

In certain embodiments, the method comprises determining the pressure of the anatomical cavity in a time less than about 60 seconds from commencing sensor measurements.

From a further aspect, there is provided a system for determining a pressure within an anatomical cavity of a patient, the system comprising: a vessel, the vessel having a first end and a second end, the first end being open and arranged to contact skin associated with the anatomical cavity of the patient to encase a portion of the skin, wherein the vessel is arranged such that a pressure can be applied through the vessel to the encased portion of the skin; a processor arranged to execute a method, the method comprising: obtaining, by the processor, a pressure value, the pressure value comprising a pressure applied to the encased portion of the skin by the vessel; obtaining, by the processor, a skin displacement value associated with the obtained pressure value, the skin displacement value comprising a distance of the encased portion of the skin from a baseline while the pressure is being applied; determining, by the processor, a pressure of the anatomical cavity using the obtained pressure value and the obtained skin displacement value, and based on a static force balance of the vessel and the encased portion of the skin while the pressure is being applied.

From a yet further aspect, there is provided a device for determining a pressure within an anatomical cavity of a patient, the device comprising: a vessel, the vessel having a first end and a second end, the first end being open and arranged to contact skin associated with the anatomical cavity of the patient to encase a portion of the skin, wherein the vessel is arranged such that a pressure can be applied through the vessel to the encased portion of the skin; a pressure unit fluidly connectable to the vessel for applying the pressure to the encased portion of the skin.

In certain embodiments, the pressure is a negative pressure and the pressure unit is fluidly connected to the second end of the vessel through which fluid can be drawn out of the vessel to apply the negative pressure to the encased portion of the skin.

In certain embodiments, the pressure unit is arranged to apply a pressure to the encased portion of the skin of between about −300 mmHg and about 300 mmHg, or between about −6 psi and about 6 psi.

In certain embodiments, the vessel has an internal diameter at the first end of about 5-10 cm. In certain embodiments, the internal diameter is more than about 1 cm, more than about 2 cm, more than about 3 cm, between about 1 cm and about 30 cm, between about 2 cm and about 30 cm, or between about 3 cm and about 30 cm.

In certain embodiments, the vessel has an internal diameter at the first end which can be modulated by a shutter-like mechanism.

In certain embodiments, there is provided a sleeve which is removably attachable sleeve to the first end of the vessel for modulating one or more of a diameter of the first end of the vessel, a shape of the first end of the vessel or a volume of the vessel.

In certain embodiments, the vessel has a length which can be modulated by a telescoping-like mechanism.

In certain embodiments, the vessel and the pressure unit are encased in an outer casing, the outer casing having a cap portion for closing a second end of the outer casing.

In certain embodiments, the device further comprises a panel attached to the outer casing for user interaction.

In certain embodiments, a length of the vessel is at least equal to, or more than, an internal radius of the vessel.

In certain embodiments, the device further comprises a pressure sensor for measuring the applied pressure in the vessel, the pressure sensor communicatively connected to the processor.

In certain embodiments, the device further comprises a distance sensor for measuring the displacement of the skin while the pressure is being applied, the distance sensor being communicatively connected to the processor.

From a further aspect, there is provided a device for determining a pressure within an anatomical cavity of a patient, the system comprising: a vessel having a cylindrical form, the vessel having a first end and a second end, the first end being open and arranged to contact skin associated with the anatomical cavity of the patient to encase a portion of the skin, wherein the vessel is arranged such that a pressure can be applied through the vessel to the encased portion of the skin; wherein an internal diameter of the vessel at the first end is between about 1 cm and about 30 cm; and a length of the vessel is at least equal to, or more than, an internal radius of the vessel.

In certain embodiments, the vessel has an internal diameter at the first end of about 5-10 cm. In certain embodiments, the internal diameter is more than about 1 cm, more than about 2 cm, more than about 3 cm, between about 1 cm and about 30 cm, between about 2 cm and about 30 cm, or between about 3 cm and about 30 cm.

In certain embodiments, the vessel has an internal diameter at the first end which can be modulated by a shutter-like mechanism.

In certain embodiments, the vessel includes a sleeve which is removably attachable sleeve to the first end of the vessel for modulating one or more of a diameter of the first end of the vessel, a shape of the first end of the vessel or a volume of the vessel.

In certain embodiments, the vessel has a length which can be modulated by a telescoping-like mechanism.

In certain embodiments, the vessel and the pressure unit are encased in an outer casing, the outer casing having a cap portion for closing a second end of the outer casing.

In certain embodiments, the device further comprises a panel attached to the outer casing for user interaction.

In certain embodiments, a length of the vessel is at least equal to, or more than, an internal radius of the vessel.

In certain embodiments, the device further comprises a pressure sensor for measuring the applied pressure in the vessel, the pressure sensor communicatively connected to the processor.

In certain embodiments, the device further comprises a distance sensor for measuring the displacement of the skin while the pressure is being applied, the distance sensor being communicatively connected to the processor.

In certain embodiments, the device is configured as a wearable device and can be mounted to the patient. The device, in such embodiments, can be used to monitor the patient over extended time periods such as hours and days. The patient could be monitored during normal activities.

From another aspect, there is provided a kit comprising a device as described herein and one or more sleeves attachable to the vessel for adapting a volume of the vessel, a diameter of the first end of the vessel, or a shape of the first end of the vessel.

According to another broad aspect of the present technology, there is provided a system comprising at least one processor and memory storing a plurality of executable instructions. When executed by the at least one processor, the executable instructions cause the system to execute the method as claimed herein.

Uses of certain embodiments of the present technology include measurement of physiological parameters, such as pressure, in a physiological cavity. Physiological cavities, but are not limited to, abdominopelvic cavity (“abdomen”), the thoracic cavity, the cranial cavity, the vertebral cavity, the pericardial cavity, the pleural cavity, muscular cavities and the mediastinum of the patient.

In certain embodiments of the present technology, the present technology can be used to measure pressure in a cavity or other enclosed volume and which is not a physiological cavity, for example, tires, balloons, etc.

Advantageously, certain embodiments of the present technology provide a non-destructive and non-invasive manner of measuring parameters such as pressure. In the case of measuring pressure in an anatomical cavity, a measure of the anatomical cavity pressure can be obtained without implanting any devices in the patient, or puncturing the patient's skin. Not only is this less painful for the patient, but the risk of infection or other complication is also reduced. Furthermore, in certain embodiments, at least a portion of the system, such as the vessel, is a handheld, portable device. The processor may also be handheld and portable especially if implemented in a portable computer system such as a mobile phone, a tablet, or the like.

Advantageously, the pressure of the anatomical cavity can be determined by applying any of the above methods in a time less than about 60 seconds.

In certain embodiments, the device is arranged to continuously monitor a patient. In this way, the cavity pressure can be determined dynamically and as the patient goes about their activities.

Various implementations of the present technology provide a non-transitory computer-readable medium storing program instructions for executing one or more methods described herein, the program instructions being executable by a processor of a computer-based system.

Various implementations of the present technology provide a computer-based system, such as, for example, but without being limitative, an electronic device comprising at least one processor and a memory storing program instructions for executing one or more methods described herein, the program instructions being executable by the at least one processor of the electronic device.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

In the context of the present specification, unless expressly provided otherwise, a computer system or computing environment may refer, but is not limited to, an “electronic device,” a “computing device,” an “operation system,” a “system,” a “computer-based system,” a “computer system,” a “network system,” a “network device,” a “controller unit,” a “monitoring device,” a “control device,” a “server,” and/or any combination thereof appropriate to the relevant task at hand.

In the context of the present specification, unless expressly provided otherwise, any of the methods and/or systems described herein may be implemented in a cloud-based environment, such as, but not limited to, a Microsoft Azure environment, an Amazon EC2 environment, and/or a Google Cloud environment.

In the context of the present specification, unless expressly provided otherwise, the expression “computer-readable medium” and “memory” are intended to include media of any nature and kind whatsoever, non-limiting examples of which include RAM, ROM, disks (e.g., CD-ROMs, DVDs, floppy disks, hard disk drives, etc.), USB keys, flash memory cards, solid state-drives, and tape drives. Still in the context of the present specification, “a” computer-readable medium and “the” computer-readable medium should not be construed as being the same computer-readable medium. To the contrary, and whenever appropriate, “a” computer-readable medium and “the” computer-readable medium may also be construed as a first computer-readable medium and a second computer-readable medium.

These and other aspects and features of non-limiting embodiments will now become apparent to those skilled in the art upon review of the following description of specific non-limiting embodiments in conjunction with the accompanying drawings.

### BRIEF DESCRIPTION OF DRAWINGS

Further aspects and advantages of the present invention will become better understood with reference to the description in association with the following in which:

FIG. 1A is schematic of a system, including a vessel, a pressure unit and a processor, for determining a physiological parameter associated with an anatomical cavity, according to certain embodiments of the present technology;

FIG. 1B is a schematic of a vessel and pressure unit, for use in the system of FIG. 1A, according to certain embodiments of the present technology;

FIGS. 2A and 2B are exploded and non-exploded perspective views, respectively of a vessel and a pressure unit portion of the system of FIG. 1, according to certain embodiments of the present technology;

FIGS. 3A and 3B are perspective opaque and translucent views, respectively, of the vessel of FIG. 1A or 1B, according to certain embodiments of the present technology;

FIGS. 4A and 4B are perspective opaque and translucent views, respectively, of a housing portion of the pressure unit of the system of FIG. 1A or 1B, according to certain embodiments of the present technology;

FIGS. 5A and 5B are perspective opaque and translucent views, respectively, of a cap portion of the pressure unit of the system of FIG. 1A or 1B, according to certain embodiments of the present technology;

FIGS. 6A and 6B are perspective opaque and translucent views, respectively, of an alternative embodiment of the vessel and pressure unit portion of FIGS. 2A and 2B and depicting a cap portion, a pump, a distance sensor, a pressure sensor and a microcontroller, according to certain embodiments of the present technology;

FIG. 7A is perspective view of the pressure unit of FIGS. 6A and 6B with the pump and the cap portion omitted, and FIG. 7B is a perspective view of the cap portion of FIGS. 6A and 6B, according to certain embodiments of the present technology;

FIG. 8 is a schematic of a computer system for executing a method of the present technology, according to certain embodiments of the present technology;

FIG. 9 is a flow diagram of a method for determining a physiological parameter associated with an anatomical cavity, according to certain embodiments of the present technology;

FIG. 10 is a schematic of a model used to determine the anatomical pressure, according to certain embodiments of the present technology; and

FIGS. 11A and 11B are side and end views, respectively, of a sleeve which is attachable to the vessel of FIG. 1A, 1B, 2A, 2B, 3A, 3B, 6A, 6B, 7A, according to certain embodiments of the present technology.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Reference will now be made in detail to various non-limiting embodiments of attachment systems for endoscopes. It should be understood that other non-limiting embodiments, modifications and equivalents will be evident to one of ordinary skill in the art in view of the non-limiting embodiments disclosed herein and that these variants should be within scope of the appended claims.

Furthermore, it will be recognized by one of ordinary skill in the art that certain structural and operational details of the non-limiting embodiments discussed hereafter may be modified or omitted (i.e. non-essential) altogether. In other instances, well known methods, procedures, and components have not been described in detail.

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements.

Broadly, there is provided a system 10 for determining a physiological parameter, such as pressure, associated with an anatomical cavity of a patient. The system is non-invasive. The system and method will be described below with reference to determination of the physiological parameter of “pressure” within the anatomical cavity, but it will be appreciated that the system and method can be used to determine other physiological parameters such as tissue elasticity and parameters derivable from pressure and tissue elasticity. The system could also be applied to non-physiological systems such as pressure measurement in enclosed volumes not associated with the patient. Broadly the system 10 is arranged to determine the physiological parameter based on displacement properties of skin adjacent the anatomical cavity during application of a pressure to the skin. In certain embodiments, the anatomical cavity is the abdominopelvic cavity. The parameter to be determined is a pressure within the abdominopelvic cavity.

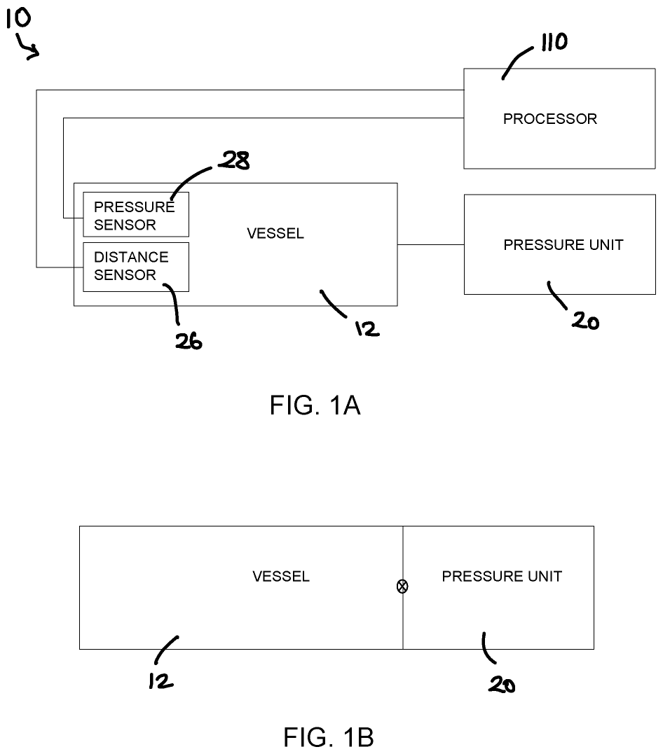

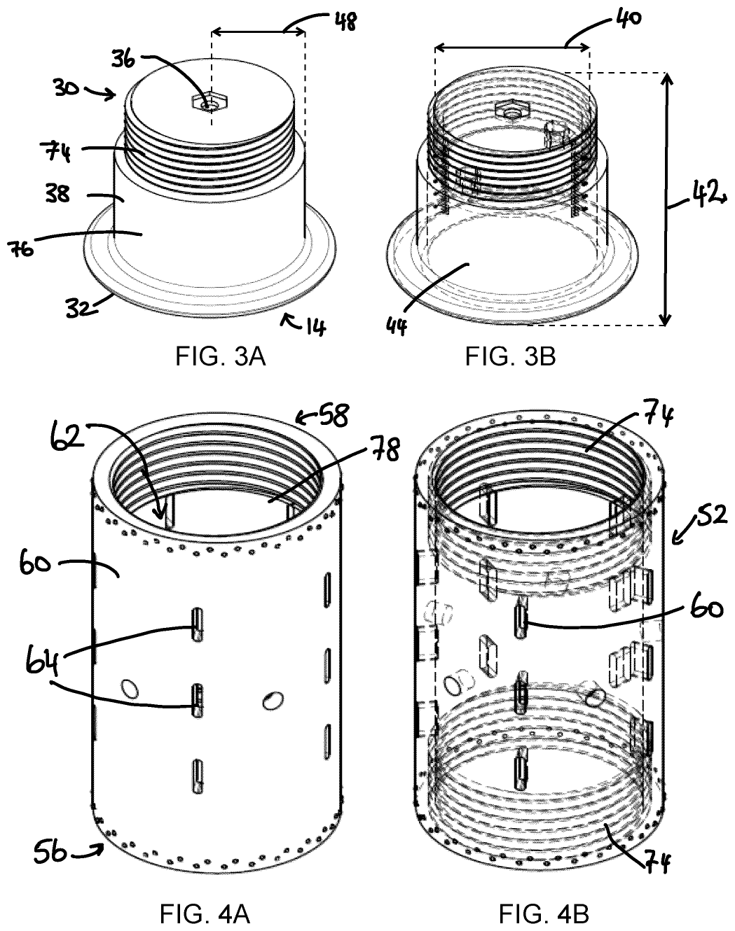

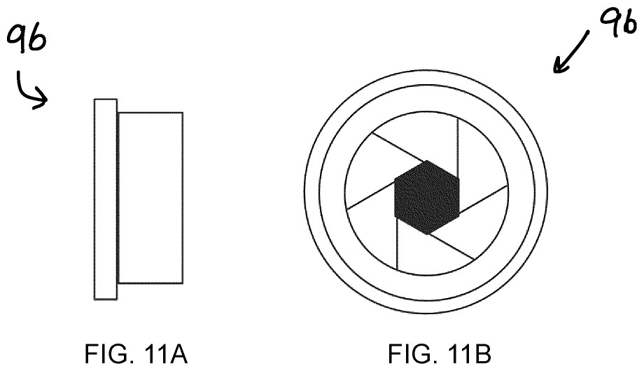

FIG. 1 is a block diagram of the system 10 in accordance with various embodiments of the present technology. The system 10 comprises a vessel 12 having an open end 14 (best seen in FIGS. 3B and 6b) which can be cupped over skin 16 associated with the anatomical cavity to encase a portion of the skin (“encased skin”) 18. The vessel 12 is arranged to accommodate an applied pressure therein which in turn applies the pressure to the encased skin 18 in use. The system 10 is provided, in certain embodiments, with a pressure unit 20 fluidly connected to the vessel 12 to modulate the pressure inside the vessel 12. In use, pressure inside the vessel 12 cupped over the encased skin 18 causes a displacement 22 of the encased skin 18 from a baseline 24 which can be detected using for example a distance sensor 26 (FIG. 6B). By displacement 22 is meant the encased tissue's 18 peak vertical displacement, in certain embodiments (FIG. 10). The applied pressure to the encased skin 18 is measured, in certain embodiments by a pressure sensor 28 (FIG. 6B). The applied pressure can be negative or positive. In the case of an applied negative pressure, the displacement 22 of the encased skin 18 is upwardly from the open end 14 of the vessel 12 towards a closed end 30 of the vessel 12.

A processor 110 of a computer environment 100 is provided, in certain embodiments, to determine the pressure in the anatomical cavity based on the determined displacement 22 of the encased skin 18 and the applied pressure. The processor 110 may be communicatively coupled to one or more of the pressure unit 20, the distance sensor 26 and/or the pressure sensor 28, and will be described later with reference to FIG. 8.

The processor 110 may be configured to receive data from the one or more of the pressure unit 20, the distance sensor 26 and/or the pressure sensor 28. In this respect, in wireless configurations, the vessel 12 may be provided with a transmitter for wirelessly transmitting the data to the processor 110. In other, wired, configurations, the data may be transmitted to the processor 110 by a wired connection. In certain embodiments, the vessel 12 may include a power source, such as a battery, for providing power to one or more of the pressure unit 20, the distance sensor 26, the pressure sensor 28 and/or the transmitter if present. The transmitter may be incorporated within a microcontroller housed in the pressure unit 20.

The vessel 12 will now be described in further detail and with specific reference to FIGS. 3A and 3B. The open end 14 of the vessel 12 is also referred to as a first end 14, and the closed end 30 of the vessel 12 is also referred to as a second end 30. The open end 14 is arranged to be brought into contact with the skin 16 adjacent the anatomical cavity of the patient. In other words, the vessel 12 may be brought into contact with the anatomical cavity via the skin. A rim 32 at the open end 14 has a lip 34 for comfort to the patient when the open end 14 is made to contact the skin 16. The lip 34 may be omitted in certain embodiments, or vary from the configuration illustrated herein. The closed end 30 has a closed end opening 36 through which fluid can be caused to flow to modulate a pressure within the vessel 12. The fluid is air, but in other embodiments can also be another gas or even a liquid.

The vessel 12 has a vessel body 38 which has a substantially cylindrical form and a substantially constant internal diameter 40 along its length 42, in certain embodiments. In certain other embodiments, the vessel may have a circular first end 14 and a conical or curvilinear overall configuration.

The vessel 12 can be considered as cup-like with the vessel body 38 defining an internal space 44 within which the pressure can be modulated by the pressure unit 20. As illustrated, in certain embodiments the first end 14 of the vessel has a circular form. It will be appreciated that the configuration of the first end 14 is not limited and it may have any other configuration such as quadrilateral, triangular, etc.

The internal diameter 40 of the vessel 12 is more than about 1 cm, more than about 2 cm, or more than about 3 cm. In certain embodiments, the internal diameter of the vessel 12 is between about 1 cm and about 30 cm, between about 2 cm and about 30 cm, or between about 3 cm and about 30 cm. The length 42 of the vessel 12 is at least equal to, or more than, an internal radius 48 of the vessel 12, in certain embodiments.

A wall thickness of the vessel 12 is such that the wall can resist deformation whilst limiting a weight of the vessel. In certain embodiments, the wall thickness of the vessel 12 is about 3 mm to about 5 mm.

The body 38 of the vessel 12 may be made of an opaque (FIG. 3A), or a translucent or transparent (FIG. 3B) material. In the case of a translucent body 38, the system 10 may dispense with the need for a distance sensor 26 as a user of the system 10 can manually observe and measure the displacement 22 of the encased skin 18 through the vessel body 38. Accordingly, the vessel 12 may have gradations (not shown) along at least a portion of the length 42 its vessel body 38 for ease of measuring the displacement 22 of the encased skin 18.

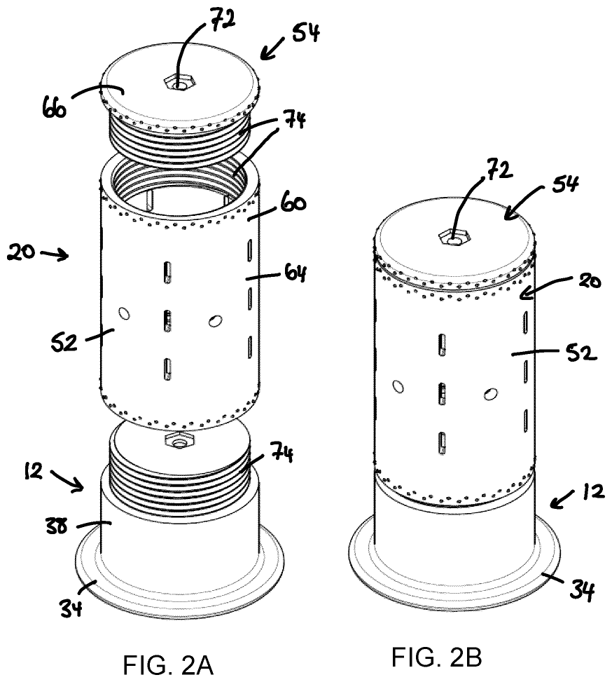

As best seen in FIGS. 2A, 2B, 4A, 4B, 5A and 5B, in certain embodiments, the pressure unit 20 comprises a housing portion 52 and a cap portion 54. The housing portion 52 has a first housing end 56 and a second housing end 58. The housing portion 52 is arranged to connect to the closed end 30 of the vessel 12 by the first housing end 56. The first housing end 56 and the second housing end 58 are both open. The housing portion 52 has a housing body 60 which is cylindrical and defines a housing channel 62. Optionally, housing openings 64 are provided in the housing body 60 for allowing the user to observe inside the housing portion 52. The housing portion 52 is arranged to enclose inner components and/or to function as an adaptor between the vessel 12 and a pump (not shown). The pump may be a hand-held pump such as one that comprises a rubber bulb, compression and release of which causes a pressure differential. In other embodiments, the pump may be a motorized pump. The pump may be operated by the processor 110.

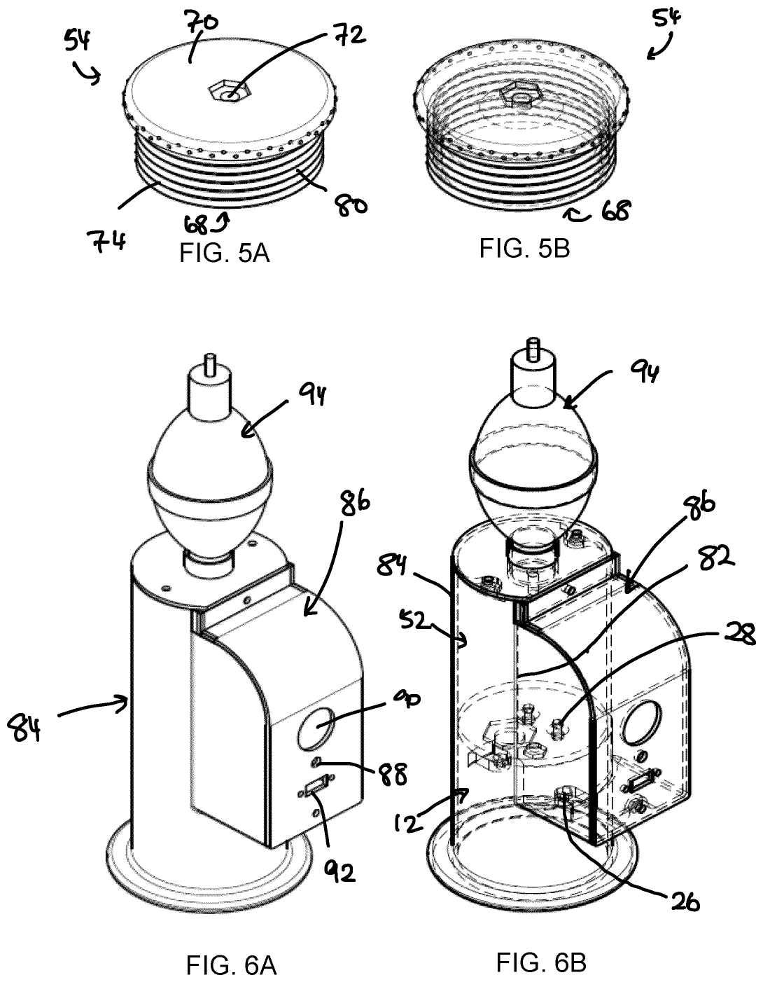

Turning now to the cap portion 54 best seen in FIGS. 5A and 5B, the cap portion 54 has a cap body 66, a first cap end 68, and a second cap end 70, the first cap end 68 being arranged to connect to the second housing end 58. The first cap end 68 is open and the second cap end 70 is closed. A cap opening 72 is provided in the second cap end 70. The cap opening 72 is smaller than a diameter of the first cap end 68.

The vessel 12, the housing portion 52 and the cap portion 54 are arranged to be connected together by a screw mechanism, and accordingly include threads 74 at the closed end 30 of the vessel 12, the first housing end 56, the second housing end 58, and the first cap end 68. The threads 74 at the closed end 30 of the vessel 12 are on an outer side 76 of the vessel body 38. The threads 74 of the housing portion 52 are on an inner side 78 of the housing body 60. The threads 74 on the cap portion 54 are on an outer side 80 of a cap body 66. The housing portion 52, the cap portion 54 and the vessel 12 are sized such that the threads 74 at the closed end 30 of the vessel 12 and the threads 74 at the first cap end 68 can be received into the housing body 60 when assembled. In alternative embodiments, instead of a screw mechanism for connecting the vessel 12, the housing portion 52 and the cap portion 54, any other type of mechanism for connecting the pieces may be provided such as screws, clips, and the like.

The vessel 12 and/or the housing portion 52 may also be configured to include one or more signalling elements for indicating one or more messages to a user, such as a power level, a data delivery confirmation or error, a pressure, a pressure error, a leak. The one or more signalling elements may include a light signal emitter (e.g. LEDs), a sound signal emitter (e.g. a speaker), or a display. One or more actuatable buttons may be provided to turn and off a power, or to reset. A relief valve may be included for pressure release.

In use, the vessel 12, the housing portion 52 and the cap portion 54 are assembled as one piece. These components may also be referred to as a device. The vessel 12 is positioned on the patient's skin. Lubricant may be used to obtain or improve a seal between the lip 34 of the vessel 12 and the skin. The pump is fluidly connected to the cap portion 54 and can cause fluid to travel through the cap opening 72, through the housing channel 62 of the housing portion 52 and into the vessel 12 through the closed end opening 36 of the vessel 12, to modulate the pressure within the vessel 12. In certain embodiments, the pump is configured to decrease the pressure within the vessel 12. The processor 110 may be configured to control the pump. In certain embodiments, the processor 110 may control the pump based on data obtained from the pressure sensor until a predetermined pressure in the vessel 12 is obtained. The distance sensor 26 and/or the pressure sensor 28 are configured to measure distance and/or pressure, respectively, before, during or after the application of pressure. The processor 110 is configured to receive data from the distance sensor 26 and/or the pressure sensor 28.

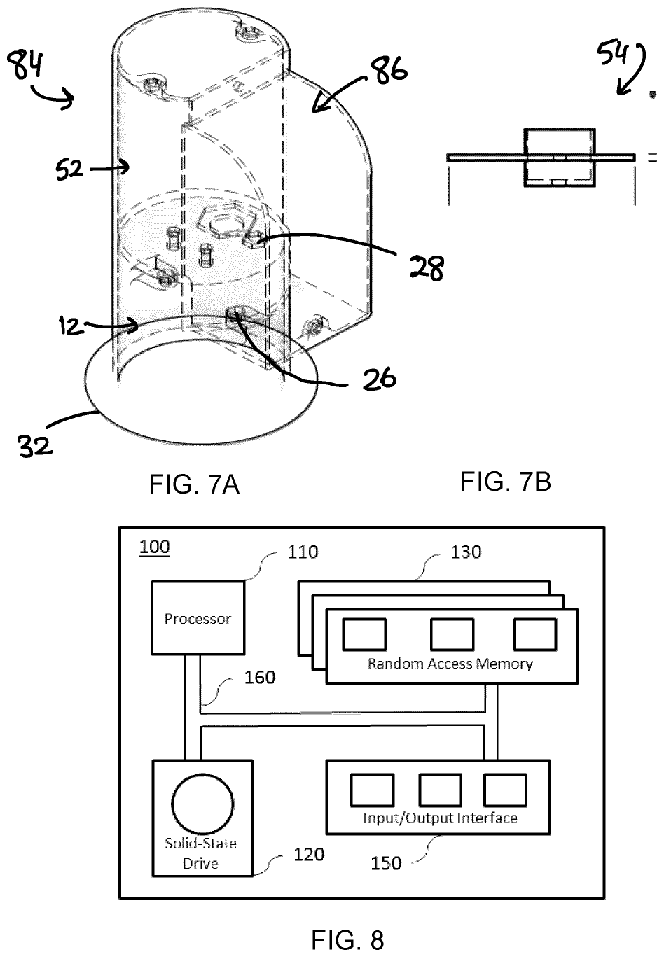

Turning now to FIGS. 6A, 6B, 7A and 7B, an alternative embodiment of the vessel 12 and pressure unit 20 is illustrated in which the housing portion 52 of the pressure unit 20 and the vessel 12 are mounted within an outer casing 84, and the cap portion 54 is configured to seal an open end of the outer casing 84. A panel 86 is provided on the outer casing 84 which includes a signaling elements in the form of an LED 88 indicating power on/off. A power on/off switch 90 is also provided, as well as a USB cable port 92. The pump is a handheld pressure bulb 94.

In yet other embodiments (not shown), the vessel 12 may have other form factors which differ from that as described and illustrated herein. For example, instead of having a cylindrical configuration, the vessel 12 may be conical.

In yet further embodiments, the vessel 12 may be configured to have an adjustable configuration such that a size of the first end 14 or a shape of the first end 14 may be modulated.

As shown in FIG. 11A, in certain embodiments, one or more sleeves 96 may be provided which are removably attachable to the first end 14 of the vessel 12 and configured to alter a volume of the vessel 12, or to alter a shape and/or a diameter of the first end 14 of the vessel 12. The one or more sleeves 96 may be provided as part of a kit.

As shown in FIG. 11B, in certain embodiments, the first end 14 of the vessel 12, or the sleeve 96 attachable to the first end 14 of the vessel, may be configurable to change shape and size through spiral shutter-type mechanism. A locking mechanism may be provided to lock a given shape and/or size. In certain other embodiments, the vessel 12 may include a telescoping mechanism configured to modulate the length 42 of the vessel. A locking mechanism may be provided to lock a given length.

In further embodiments (not shown), the pressure unit 20 may differ from that as illustrated and described herein in that the pressure differential in the vessel 12 may be created by a change in volume. In these embodiments, the pressure unit 20 may have a deformable configuration permitting a compressed form in which its volume is reduced, and a released form which has a higher volume. Applying the vessel 12 and pressure unit 20 to the skin when the pressure unit 20 is in its compressed form, then allowing the pressure unit 20 to expand, would create drop in pressure in the vessel 12 thereby causing the skin displacement.

In yet further embodiments, at least the vessel 12 and the housing portion 52 of the pressure unit 20 may have a wearable configuration. Such embodiments may be used to monitor a patient over a given time frame, such as hours, days or weeks. The wearable configuration may include a strap for mounting to the patient, or adhesive, or the like.

Turning now to the computing environment illustrated in FIG. 8, which may be used to implement and/or execute any of the methods described herein. In some embodiments, the computing environment 100 may be implemented by any of a conventional personal computer, a network device and/or an electronic device (such as, but not limited to, a mobile device, a tablet device, a server, a controller unit, a control device, etc.), and/or any combination thereof appropriate to the relevant task at hand. In some embodiments, the computing environment 100 comprises various hardware components including one or more single or multi-core processors collectively represented by processor 110, a solid-state drive 120, a random access memory 130, and an input/output interface 150. The computing environment 100 may be a computer specifically designed to operate a machine learning algorithm (MLA). The computing environment 100 may be a generic computer system.

In some embodiments, the computing environment 100 may also be a subsystem of one of the above-listed systems. In some other embodiments, the computing environment 100 may be an “off-the-shelf” generic computer system. In some embodiments, the computing environment 100 may also be distributed amongst multiple systems. For example, a microcontroller 82 may be provided (FIG. 7) within the vessel for collecting values of the displacement 22 and the applied pressure, and wirelessly sending that to an external processor for the determination step. The computing environment 100 may also be specifically dedicated to the implementation of the present technology. As a person in the art of the present technology may appreciate, multiple variations as to how the computing environment 100 is implemented may be envisioned without departing from the scope of the present technology.

Those skilled in the art will appreciate that processor 110 is generally representative of a processing capability. In some embodiments, in place of or in addition to one or more conventional Central Processing Units (CPUs), one or more specialized processing cores may be provided. For example, one or more Graphic Processing Units (GPUs), Tensor Processing Units (TPUs), and/or other so-called accelerated processors (or processing accelerators) may be provided in addition to or in place of one or more CPUs.

System memory will typically include random access memory 130, but is more generally intended to encompass any type of non-transitory system memory such as static random access memory (SRAM), dynamic random access memory (DRAM), synchronous DRAM (SDRAM), read-only memory (ROM), or a combination thereof. Solid-state drive 120 is shown as an example of a mass storage device, but more generally such mass storage may comprise any type of non-transitory storage device configured to store data, programs, and other information, and to make the data, programs, and other information accessible via a system bus 160. For example, mass storage may comprise one or more of a solid state drive, hard disk drive, a magnetic disk drive, and/or an optical disk drive.

Communication between the various components of the computing environment 100 may be enabled by a system bus 160 comprising one or more internal and/or external buses (e.g., a PCI bus, universal serial bus, IEEE 1394 “Firewire” bus, SCSI bus, Serial-ATA bus, ARINC bus, etc.), to which the various hardware components are electronically coupled.

The input/output interface 150 may allow enabling networking capabilities such as wired or wireless access. As an example, the input/output interface 150 may comprise a networking interface such as, but not limited to, a network port, a network socket, a network interface controller and the like. Multiple examples of how the networking interface may be implemented will become apparent to the person skilled in the art of the present technology. For example the networking interface may implement specific physical layer and data link layer standards such as Ethernet, Fibre Channel, Wi-Fi, Token Ring or Serial communication protocols. The specific physical layer and the data link layer may provide a base for a full network protocol stack, allowing communication among small groups of computers on the same local area network (LAN) and large-scale network communications through routable protocols, such as Internet Protocol (IP).

The input/output interface 150 may be coupled to a touchscreen and/or to the one or more internal and/or external buses 160. The touchscreen may be part of the display. In some embodiments, the touchscreen is the display. The touchscreen may comprise touch hardware (e.g., pressure-sensitive cells embedded in a layer of a display allowing detection of a physical interaction between a user and the display) and a touch input/output controller allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the computing device 100 in addition to or instead of the touchscreen. For example, the user may use any of the mouse, keyboard, trackpad and touchscreen to manually provide one or more of the displacement 22 of the encased skin 18, a target pressure to be applied to the encased skin, or a pressure reading within the vessel 12.

According to some implementations of the present technology, the solid-state drive 120 stores program instructions suitable for being loaded into the random access memory 130 and executed by the processor 110 for executing acts of one or more methods described herein. For example, at least some of the program instructions may be part of a library or an application.

All statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown. Moreover, it should be understood that one or more modules may include for example, but without being limitative, computer program logic, computer program instructions, software, stack, firmware, hardware circuitry, or a combination thereof.

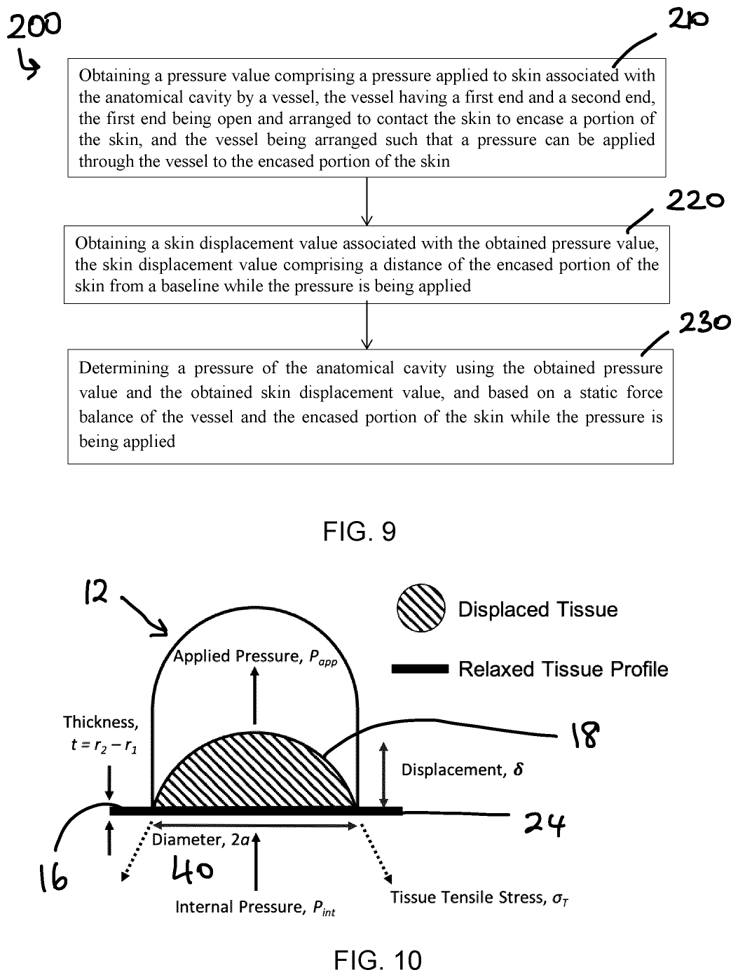

Turning now to FIG. 9 which illustrates a flow diagram of a method 200 for determining the physiological parameter associated with the anatomical cavity of the patient, in accordance with various embodiments of the present technology. The method 200 may be executed by a processor of a computer system, such as the processor 110.

At Step 210, the method 200 comprises obtaining, by the processor 110, a pressure value.

The pressure value comprises the pressure applied to the encased skin 18 by the vessel 12. The pressure value may be obtained from the pressure sensor 28 communicatively connected to the processor 110. Alternatively, the pressure value may be obtained in any other way, such as through a manual input to the processor, from the pressure unit 20, or by any other means.

At Step 220, the method 200 comprises obtaining, by the processor 110, a skin displacement value associated with the obtained pressure value. The skin displacement value comprises the displacement 22 of the encased skin 18 from the baseline 24 while the pressure is being applied to the encased skin 18. The skin displacement value may be obtained from the distance sensor 26 communicatively connected to the processor 110. Alternatively, the displacement value may be obtained in any other way, such as through a manual input to the processor, or by any other means.

At Step 230, the method 200 comprises determining, by the processor 110, a pressure of the anatomical cavity using the obtained pressure value and the obtained skin displacement value, and based on a static force balance of the vessel 12 and the encased skin 18 while the pressure is being applied. The static force balance of the vessel and the encased portion of the skin while the pressure is being applied is based on a model of a thick-walled cylinder which is a geometric simplification of the abdomen. The model is illustrated in FIG. 10. The equation below can be used to determine abdomen thick wall hoop stress:

$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )}$$

where Pᵢₙ is the internal vessel pressure, Pₐₚₚ is the applied pressure, r₁ and r₂ are the inner and outer curve radii, respectively, a is the vessel radius, w is the skin displacement value, and t is tissue thickness.

The model at body positions other than supine introduces a fluid pressure factor, such that

$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )} + ρ g h$$

where ρ is the density of the fluid, g is the force of gravity, and h is the height of the centroid of the anatomical cavity being tested.

The method 200 may further comprise the following steps:

Determining the local elasticity in a wall of the anatomical cavity is determined using:

$$E = \frac{α ( ζ , ν ) 3 ϕ ( η ) ( {P}_{atm} - {P}_{a p p} ) a}{2 π w}$$

where E is Young's Modulus, a(ζ,v) is a coefficient dependant on the ratio of tissue thickness to vessel inner radius (ζ=t/r₁) and Poisson's ratio (v), ϕ(η) is a geometric coefficient, and Pₐₜₘ is atmospheric pressure. (Theret, D. et al 1988. J. Biomech. Eng.; Transactions of the ASME 110(3): 190-199; Boudou et al 2006, J. Biomechanics 39(9):1677-1685; the contents of which are incorporated herein by reference.

In certain other embodiments, the processor 110 may be configured to obtain data from other physiological parameters associated with the tissue. For example, bioimpedance data or ultrasound data for determining one or both of tissue thickness, elasticity or capacitance. For example, in certain embodiments, bioimpedance measures of the skin can be used to determine elasticity, instead of the method described above.

In certain embodiments, the method 200 further comprises causing by the processor 110, the pressure unit 20 fluidly connected to the vessel 12 to modulate the pressure in the vessel 12, when the vessel is encasing the skin 18, such that the applied pressure to the encased skin 18 is between about −300 mmHg and about 300 mmHg, or between about −6 psi and about 6 psi. In certain embodiments, the applied pressure is about 5 psi.

In certain embodiments, the method 200 further comprises causing the pressure unit 20 to apply the applied pressure for a time less than about 60 seconds. In other embodiments, the method 200 is configured to monitor the patient over longer time periods, such as during at least a portion of a day, week or month.

In certain embodiments, the method 200 further comprises causing, by the processor 110, the vessel 12 to contact the skin before the pressure is applied. In this case, the system 10 may be provided with a robot arm (not shown) for holding and moving the vessel. Alternatively, the vessel 12 may be positioned manually by the user of the system 10 or by the patient. In certain embodiments in which the anatomical cavity is the abdomen, the vessel is positioned at approximately 5 cm subxiphoid.

### Example

The system 10 was applied to Patient X. Patient X had an abdominal wall thickness of 30 mm, an abdominal circumference at the navel of 80 cm, and an TAP of 5 mmHg (0.667 kPa). The vessel 12, having a radius, a, of 30 mm was applied to abdominal skin associated with an abdomen of Patient X along the linea alba. A portion of the skin was encased by the vessel 12. A negative pressure of about 15 mmHg (2.0 kPa) was applied through the vessel 12 to the encased portion of the skin. A skin displacement value of approximately 1 mm was obtained.

The pressure within the abdomen of the patient was determined as follows:

$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )} = \frac{( 2.00 ) ( 0.0 {3}^{2} + 0.0 {1}^{2} ) ( 0.12 {7}^{2} - 0.09 {7}^{2} )}{4 ( 0.03 ) ( 0.01 ) ( 0.12 {7}^{2} + 0.09 {7}^{2} ) - ( 0.0 {3}^{2} + 0.0 {1}^{2} ) ( 0.12 {7}^{2} - 0.09 {7}^{2} )} = 0.5617 kPa or 4.213 mmHg$$

where r₂ and r₁ were calculated, geometrically, by abdominal circumference (C)

$${r}_{2} \frac{C}{2 π} , {r}_{1} = {r}_{2} - t$$

It was found that measured TAP was within an acceptable range of the calculated TAP. The device and method described herein could identify the correct range of TAP in vivo (low, mid, high).

Also,

$$E = \frac{α ( ζ , ν ) 3 ϕ ( η ) ( {P}_{atm} - {P}_{a p p} ) a}{2 π w} = \frac{0.117 ( 6.8989 ) ( 101.3 - 2.0 ) ( 0.03 )}{2 π ( 0.01 )} = 38.27 kPa$$

This value is within reason, given experimental results indicating Young's Modulus, E, at the linea alba is wide ranging (28.71-42.5 kPa) due to patient to patient variation.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

It should be appreciated that the invention is not limited to the particular embodiments described and illustrated herein but includes all modifications and variations falling within the scope of the invention as defined in the appended claims.

1. A method for determining a pressure within an anatomical cavity of a patient, the method being executed by a processor, the method comprising;
obtaining, by the processor, a pressure value, the pressure value comprising a pressure applied to skin associated with the anatomical cavity by a vessel, the vessel having a first end and a second end, the first end being open and arranged to contact the skin to encase a portion of the skin, and the vessel being arranged such that a pressure can be applied through the vessel to the encased portion of the skin;
obtaining, by the processor, a skin displacement value associated with the obtained pressure value, the skin displacement value comprising a distance of the encased portion of the skin from a baseline while the pressure is being applied;
determining, by the processor, a pressure of the anatomical cavity using the obtained pressure value and the obtained skin displacement value, and based on a static force balance of the vessel and the encased portion of the skin while the pressure is being applied.

2. The method of claim 1, wherein the static force balance of the vessel and the encased portion of the skin while the pressure is being applied is based on a model of a thick-walled cylinder.

3. The method of claim 2, wherein the model is defined for a supine anatomical cavity as:
$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )}$$
where Pᵢₙ is the internal vessel pressure, Pₐₚₚ is the applied pressure, r₁ and r₂ are the inner and outer curve radii, respectively, a is the vessel radius, w is the skin displacement value, and t is tissue thickness;
and for a non-supine anatomical cavity as:
$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )} + ρ g h$$
where ρ is the density of the fluid, g is the force of gravity, and h is the height of the centroid of the anatomical cavity being tested.

4. The method of claim 3,
wherein the local elasticity in a wall of the anatomical cavity is determined using:
$$E = \frac{α ( ζ , ν ) 3 ϕ ( η ) ( {P}_{atm} - {P}_{a p p} ) a}{2 π w}$$
where E is Young's Modulus, a(ζ,v) is a coefficient dependant on the ratio of tissue thickness to vessel inner radius (ζ=t/r₁) and Poisson's ratio (v), ϕ(η) is a geometric coefficient, and Pₐₜₘ is atmospheric pressure.

5. The method of any of claims 1-4, wherein obtaining the pressure value comprises the processor obtaining data from a pressure sensor measuring pressure within the vessel and communicatively connected to the processor.

6. The method of any of claims 1-5, wherein obtaining the skin displacement value comprises the processor obtaining data from a distance sensor measuring the displacement of the encased portion of the skin in the vessel and communicatively connected to the processor.

7. The method of any of claims 1-6, wherein the determining the pressure of the anatomical cavity is based on the applied pressure to the encased portion of the skin being between −300 mmHg and 300 mmHg, or between −6 psi and 6 psi.

8. The method of any of claims 1-7, further comprising causing, by the processor, a pressure unit connected to the vessel to apply the pressure to the encased portion of the skin through the vessel.

9. The method of claim 8, wherein the applied pressure is between about −300 mmHg and about 300 mmHg, or between about −6 psi and about 6 psi.

10. The method of any of claims 1-9, further comprising causing, by the processor, the vessel to contact the skin before the pressure is applied.

11. The method of any of claims 1-10, wherein the anatomical cavity is an abdomen of a patient.

12. The method of any of claims 1-11, wherein the applied pressure is a negative pressure applied through an opening at the second end of the vessel, and the skin displacement comprises a distance of the encased portion of the skin from the baseline towards the second end of the vessel.

13. The method of any of claims 1-12, wherein the applied pressure is a positive pressure.

14. A system for determining a pressure within an anatomical cavity of a patient, the system comprising a processor arranged to execute a method, the method comprising:
obtaining, by the processor, a pressure value, the pressure value comprising a pressure applied to an encased portion of skin of the patient associated with the anatomical cavity through a vessel, the vessel having a first end and a second end, the first end being open and arranged to contact the skin to encase a portion of the skin, wherein the vessel is arranged such that a pressure can be applied through the vessel to the encased portion of the skin;
obtaining, by the processor, a skin displacement value associated with the obtained pressure value, the skin displacement value comprising a distance of the encased portion of the skin from a baseline while the pressure is being applied;
determining, by the processor, a pressure of the anatomical cavity using the obtained pressure value and the obtained skin displacement value, and based on a static force balance of the vessel and the encased portion of the skin while the pressure is being applied.

15. The system of claim 14, further comprising the vessel.

16. The system of claim 14 or claim 15, further comprising a pressure unit fluidly connectable to the vessel for applying the pressure to the encased portion of the skin.

17. The system of claim 17, wherein the pressure unit comprises a pump.

18. The system of claim 16 or claim 17, wherein the pressure is a negative pressure and the pressure unit is fluidly connected to the second end of the vessel through which fluid can be drawn out of the vessel to apply the negative pressure to the encased portion of the skin.

19. The system of any of claims 16-18, wherein the pressure unit is arranged to apply a pressure to the encased portion of the skin of between about −300 mmHg and about 300 mmHg, or between about −6 psi and about 6 psi.

20. The system of any of claims 14-19, wherein the vessel has an internal diameter at the first end of about 5-10 cm.

21. The system of any of claims 14-20, wherein the vessel has an internal diameter at the first end which can be modulated by a shutter-like mechanism.

22. The system of any of claims 14-21, wherein the vessel has a length which can be modulated by a telescoping-like mechanism.

23. The system of any of claims 14-22, wherein the vessel and the pressure unit are encased in an outer casing, the outer casing having a cap portion for closing a second end of the outer casing.

24. The system of claim 23, further comprising a panel attached to the outer casing for user interaction.

25. The system of any of claims 14-24, wherein a length of the vessel is at least equal to, or more than, an internal radius of the vessel.

26. The system of any of claims 14-25, further comprising a pressure sensor for measuring the applied pressure in the vessel, the pressure sensor communicatively connected to the processor.

27. The system of any of claims 14-26, further comprising a distance sensor for measuring the displacement of the skin while the pressure is being applied, the distance sensor being communicatively connected to the processor.

28. The system of any of claims 14-27, wherein the static force balance of the vessel and the skin while the pressure is being applied is based on a model of a thick-walled cylinder.

29. The system of claim 28, wherein the model is defined for a supine anatomical cavity as:
$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )}$$
where Pᵢₙ is the internal vessel pressure, Pₐₚₚ is the applied pressure, r₁ and r₂ are the inner and outer curve radii, respectively, a is the vessel radius, w is the skin displacement value, and t is tissue thickness;
and for a non-supine anatomical cavity as:
$${P}_{i n} = \frac{( {P}_{a p p} ) ( {a}^{2} + {w}^{2} ) ( {r}_{2}^{2} - {r}_{1}^{2} )}{4 t w ( {r}_{1}^{2} + {r}_{2}^{2} ) - ( {a}^{2} + {w}^{2} ) ( {r}_{2}^{2} - {r}_{1}^{2} )} + ρ g h$$
where ρ is the density of the fluid, g is the force of gravity, and h is the height of the centroid of the anatomical cavity being tested.

30. The method of claim 29,
wherein the local elasticity in a wall of the anatomical cavity is determined using:
$$E = \frac{α ( ζ , ν ) 3 ϕ ( η ) ( {P}_{atm} - {P}_{a p p} ) a}{2 π w}$$
where E is Young's Modulus, a(ζ,v) is a coefficient dependant on the ratio of tissue thickness to vessel inner radius (ζ=t/r₁) and Poisson's ratio (v), ϕ(η) is a geometric coefficient, and Pₐₜₘ is atmospheric pressure.

31. The system of claim 30, wherein the applied pressure is between about −300 mmHg and about 300 mmHg, or between about −6 psi and about 6 psi.

32. The system of any of claims 16-31, wherein the anatomical cavity is an abdomen of a patient.

33. The system of any of claims 16-32, wherein the applied pressure is a negative pressure applied through an opening at the second end of the vessel.

34. The system of any of claims 16-32, wherein the applied pressure is a positive pressure.

35. A device for determining a pressure within an anatomical cavity of a patient, the device comprising:
a vessel, the vessel having a first end and a second end, the first end being open and arranged to contact skin associated with the anatomical cavity of the patient to encase a portion of the skin, wherein the vessel is arranged such that a pressure can be applied through the vessel to the encased portion of the skin;
a pressure unit fluidly connectable to the vessel for applying the pressure to the encased portion of the skin.

36. The device of claim 35, wherein the pressure is a negative pressure and the pressure unit is fluidly connected to the second end of the vessel through which fluid can be drawn out of the vessel to apply the negative pressure to the encased portion of the skin.

37. The device of claim 35 or claim 36, wherein the pressure unit is arranged to apply a pressure to the encased portion of the skin of between about −300 mmHg and about 300 mmHg, or between about −6 psi and about 6 psi.

38. The device of any of claims 35-37, wherein the vessel has an internal diameter at the first end of about 5-10 cm.

39. The device of any of claims 35-38, wherein the vessel has an internal diameter at the first end which can be modulated by a shutter-like mechanism.

40. The device of any of claims 35-39, wherein the vessel has a length which can be modulated by a telescoping-like mechanism.

41. The device of any of claims 35-40, wherein the vessel and the pressure unit are encased in an outer casing, the outer casing having a cap portion for closing a second end of the outer casing.

42. The device of claim 41, further comprising a panel attached to the outer casing for user interaction.

43. The device of any of claims 35-42, wherein a length of the vessel is at least equal to, or more than, an internal radius of the vessel.

44. The device of any of claims 35-43, further comprising a pressure sensor for measuring the applied pressure in the vessel, the pressure sensor communicatively connected to the processor.

45. The device of any of claims 35-44, further comprising a distance sensor for measuring the displacement of the skin while the pressure is being applied, the distance sensor being communicatively connected to the processor.

46. A device for determining a pressure within an anatomical cavity of a patient, the device comprising:
a vessel having a cylindrical form, the vessel having a first end and a second end, the first end being open and arranged to contact skin associated with the anatomical cavity of the patient to encase a portion of the skin, wherein the vessel is arranged such that a pressure can be applied through the vessel to the encased portion of the skin;
wherein an internal diameter of the vessel at the first end is between about 1 cm and about 30 cm; and a length of the vessel is at least equal to, or more than, an internal radius of the vessel.

47. The device of claim 46, wherein the vessel has an internal diameter at the first end of about 5-10 cm.

48. The device of claim 46 or claim 47, wherein the vessel has an internal diameter at the first end which can be modulated by a shutter-like mechanism.

49. The device of any of claims 46-48, wherein the vessel has a length which can be modulated by a telescoping-like mechanism.

50. The device of any of claims 46-49, wherein the vessel and the pressure unit are encased in an outer casing, the outer casing having a cap portion for closing a second end of the outer casing.

51. The device of claim 50, further comprising a panel attached to the outer casing for user interaction.

52. The device of any of claims 46-51, wherein a length of the vessel is at least equal to, or more than, an internal radius of the vessel.

53. The device of any of claims 46-52, further comprising a pressure sensor for measuring the applied pressure in the vessel, the pressure sensor communicatively connected to the processor.

54. The device of any of claims 46-53, further comprising a distance sensor for measuring the displacement of the skin while the pressure is being applied, the distance sensor being communicatively connected to the processor.
